(19) United States
(12) Patent Application Publication
(10) Pub. No.: US 2024/0207526 A1
(43) Pub. Date: Jun. 27, 2024

(54) Coded Housing Components for an Injection Device

(71) Applicant: Sanofi, Paris (FR)

(72) Inventors: Beate Franke, Frankfurt am Main (DE); Christian Frescha, Rüsselsheim (DE); Michael Helmer, Frankfurt am Main (DE); Stephan Muecke, Rüsselsheim (DE); Peter Nober, Rüsselsheim (DE); Matthias Rau, Rüsselsheim (DE); Michael Schabbach, Frankfurt am Main (DE); Martin Vitt, Frankfurt am Main (DE)

(21) Appl. No.: 18/288,450

(22) PCT Filed: May 2, 2022
(86) PCT No.: PCT/EP2022/061642

§ 371 Date: Oct. 26, 2023

(30) Foreign Application Priority Data
May 3, 2021 (EP) 21315072.5

(51) Int. Cl. A61M 5/31

(52) U.S. Cl. CPC A61M 5/3129; A61M 2005/3142; A61M 2205/6045

(57) ABSTRACT

A coded housing of a drug delivery device includes first and second housing components, respectively including a first and second connecting ends. An insert is provided on the first or second connecting end, and a receptacle is provided on the other one of the first and second connecting ends. The insert is insertable into the receptacle for mutually fastening the first and second housing components. A fastening element is provided on the insert, a counter fastening element complementary shaped to the fastening element is provided in the receptacle, a mechanical coding is provided on the insert and includes a coding feature, and a mechanical counter coding is provided in the receptacle and includes a counter coding feature. The mechanical coding and counter coding are operable to prevent an engagement of the fastening element with the counter fastening element when the mechanical coding does not match the mechanical counter coding.

### CROSS REFERENCE TO RELATED APPLICATIONS

The present application is the national stage entry of International Patent Application No. PCT/EP2022/061642, filed on May 2, 2022, and claims priority to Application No. EP 21315072.5, filed on May 3, 2021, the disclosures of which are incorporated herein by reference.

### TECHNICAL FIELD

The present disclosure relates to the field of drug delivery devices and systems, particularly to injection devices for injecting a liquid medicament. More particularly, the present disclosure is generally directed to drug delivery devices and systems comprising a multi-component housing, wherein one housing component is configured to accommodate a medicament container, such as a cartridge and wherein another housing component is configured to accommodate a drive mechanism to operably engage with the medicament container for expelling or withdrawing a dose of the medicament.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

A patient suffering from a particular disease may require a certain amount of a medicament to either be injected via a pen-type injection syringe or infused via a pump. With respect to reusable injection or delivery devices, a patient may have to load or to replace a cartridge. Reusable injection devices typically comprise a multi-component housing. For instance, the housing may comprise a proximal housing component, such as a body and a distal housing component, such as a cartridge holder detachably connectable to the body. Once a medicament provided in a medicament container, such as a cartridge, is empty, the cartridge holder may be disconnected from the body of the injection device and the empty cartridge may be removed and replaced with a new cartridge.

Another concern may arise from cartridges being manufactured in essentially standard sizes and manufactured to comply with certain recognized local and international standards. Consequently, such cartridges are typically supplied in standard sized cartridges (e.g. 3 ml cartridges). Therefore, there may be a variety of cartridges supplied by a number of different suppliers and containing a different medicament but fitting a single drug delivery device. As just one example, a first cartridge containing a first medicament from a first supplier may fit a drug delivery device provided by a second supplier. As such, a user might be able to load an incorrect medicament into a drug delivery device and, then, dispense said medicament (such as a rapid or basal type of insulin) without being aware that the medical delivery device was perhaps neither designed nor intended to be used with such a cartridge.

As such, there is a growing desire from users, health care providers, caregivers, regulatory entities, and medical device suppliers to reduce the potential risk of a user loading an incorrect drug type into a drug delivery device. It is also desirable to reduce the risk of dispensing an incorrect medicament (or the wrong concentration of the medicament) from such a drug delivery device.

There is, therefore, a general need to physically dedicate or mechanically code a cartridge and/or cartridge holder to its drug type and design an injection device that only accepts or works with the dedication or coded features provided on or with the cartridge and/or cartridge holder so as to prevent unwanted cartridge cross use. Similarly, there is also a general need for a dedicated cartridge that allows the medical delivery device to be used with only an authorized cartridge containing a specific medicament while also preventing undesired cartridge cross use.

### SUMMARY

In one aspect the disclosure relates to a housing of a drug delivery device, in particular to a housing of an injection device, such as a handheld injection pen. The housing comprises a first housing component configured to accommodate a cartridge filled with a medicament. The first housing component comprises a first connecting end. The housing further comprises a second housing component. The second housing component is configured to accommodate a drive mechanism of the drug delivery device. Typically, the drive mechanism comprises a piston rod extending in longitudinal direction and configured to operably engage with a piston or bung of the cartridge for expelling a dose of the medicament from the cartridge.

The second housing component comprises a second connecting end. Typically, the first connecting end is connectable to the second connecting end to form or to constitute the housing of the drug delivery device. With some examples the first housing component is an elongated or tubular shaped housing component comprising the first connecting end at a longitudinal proximal end. The second housing component may be also of tubular or elongated shape. The second connecting end may be located at a distal longitudinal end of the second housing component.

There is further provided an insert on one of the first connecting end and the second connecting end. The insert is typically integrally formed with the respective first or second housing component. There is further provided a receptacle on the other one of the first connecting end and the second connecting end. The insert is insertable into the receptacle along the longitudinal direction for mutually fastening the first housing component and the second housing component and/or for forming or establishing the housing of the drug delivery device. Typically, the receptacle is provided at one of the first and second connecting ends and forms a respective connecting end. The insert is provided on the other one of the first and second connecting ends and forms a respective connecting end.

The receptacle comprises an inner cross-section sized and shaped to receive the insert therein. Typically, an inside diameter or inside cross-section of the receptacle closely matches an outside diameter or outer cross-section of the insert.

The housing further comprises a fastening element provided on the insert and a counter fastening element complementary shaped to the fastening element and provided in the receptacle. Typically, and when reaching a final assembly configuration, the fastening element engages the counter fastening element thereby fastening and fixing the first housing component to the second housing component; and vice versa.

The housing further comprises a groove that is provided on one of the insert and the receptacle. The groove extends along the longitudinal direction. Typically, the groove is linearly or straight shaped and extends exclusively in the longitudinal direction or parallel to the longitudinal direction. The housing further comprises a protrusion provided on the other one of the insert and the receptacle. The protrusion is configured, hence, the protrusion is sized and/or shaped to slide along the groove upon insertion of the insert into the receptacle. Mutual engagement of the groove and the protrusion rotationally locks the first housing component relative to the second housing component during the mutual assembly of the first and second housing components.

The housing further comprises a mechanical coding provided on the insert. The mechanical coding comprises a coding feature. The housing further comprises a mechanical counter coding provided in the receptacle and comprising a counter coding feature. The mechanical coding and the mechanical counter coding are operable to prevent an engagement of the fastening element with the counter fastening element when the mechanical coding does not match the mechanical counter coding. A mutual assembly of the first housing component and the second housing component and/or a mutual engagement of the fastening element and the counter fastening element requires that a first housing component provided with a mechanical coding is assembled with a second housing component provided with a corresponding or complementary-shaped mechanical counter coding.

Only when the mechanical coding of the insert matches the mechanical counter coding in the receptacle the fastening element and the counter fastening element will be enabled to mutually engage. With all other pairings or combinations of a mechanical coding, e.g. of a first type, with a non-matching mechanical counter coding, e.g. of a second type, the fastening element and the counter fastening element are hindered from mutually engaging. Then, a mutual assembly and/or a fixing of first and second housing components is effectively prevented.

Mutual engagement of the fastening element with the counter fastening element requires that the mechanical coding matches the mechanical counter coding. In this way, unintended cross use of a first housing component of a first drug delivery device with a second housing component of another drug delivery device can be effectively prevented.

Prevention of the mutual engagement of the fastening element with the counter fastening element can be effectively achieved in two different ways. According to some examples the mechanical coding and the non-matching mechanical counter coding are configured to prevent a complete insertion of the insert into the receptacle along the longitudinal direction. Here, and before the insert reaches a final assembly position inside the receptacle the mechanical coding mechanically engages with the non-matching mechanical counter coding thereby impeding any further longitudinal movement of the insert into the receptacle. Here, the fastening element is hindered to reach an intended final assembly position in which it could engage with the complementary-shaped counter fastening element.

With other examples the fastening element is integrated into the mechanical coding and the counter fastening element is integrated into the mechanical counter coding. Here the mechanical coding non-matching with the mechanical counter coding may allow and support that the fastening element reaches a predefined final assembly position inside the receptacle but the fastening element may be hindered to engage with the counter fastening element. With this example, the fastening element may not match the respective counter fastening element. Accordingly, the fastening element is hindered to engage the counter fastening element and a mutual fastening of the first housing component and the second housing component can be effectively impeded.

The mutual engagement of the groove and the protrusion on the insert and the receptacle provides mounting and/or mutually assembling the first housing component and the second housing component. Typically, the groove adjoins a longitudinal end face of the first connecting end or of the second connecting end. The protrusion to engage with the groove may be provided near a longitudinal end of the other one of the first connecting end and the second connecting end. In this way and right upon insertion of the insert into the receptacle the insert can be rotationally locked with respect to the receptacle. Hence, the assembly of the first and second housing components is reduced to a purely longitudinal sliding movement of the first housing component relative to the second housing component. A non-rotational assembly process, wherein the first housing component is exclusively subject to a sliding motion along the longitudinal direction relative to the second housing component is rather intuitive and easy to perform, even for patients or users suffering side effects.

According to another example the mechanical coding is defined by a cross-sectional geometry of a coding portion of a sidewall of the insert. Hence, the shape and in particular of a cross-section of the sidewall of the insert defines the mechanical coding. Only a receptacle with a complementary shaped mechanical counter coding will be operable to receive the mechanical coding portion of the sidewall of the insert. Hence, the receptacle of the housing may be correspondingly shaped such as a to receive the coding portion of the sidewall of the insert with its particular cross-sectional geometry. Typically, the outer cross-section of the coding portion of the sidewall forms the mechanical coding or defines the mechanical coding.

According to a further example the mechanical coding is defined by a cross-sectional geometry of a longitudinal end face of the insert. The longitudinal end face of the insert may coincide with the first connecting end of the first housing component. Also here, it may be only the outer circumference of the cross-sectional geometry of the longitudinal end face of the insert that governs or defines the mechanical coding. In particular, the outer cross-sectional geometry of the longitudinal end face of the insert defines the mechanical coding.

By varying the cross-sectional geometry of a longitudinal end face and/or of the insert as such provides a rather intuitive and easily recognizable mechanical coding. Here, a user or patient making use of the drug delivery device may rather easily and rather intuitively distinguish one mechanical coding from another mechanical coding. By varying the cross-sectional geometry of the insert and/or of the longitudinal end face of the insert the mechanical coding can be even haptically detected.

According to a further example the cross-sectional geometry of the coding portion of the sidewall of the insert comprises one of a circular shape, an oval shape, a triangular shape, a rectangular shape and a polygonal shape. With some examples the cross-sectional geometry of the coding portion and/or the cross-section of the end face of the insert may comprise a pentagonal, hexagonal, or even octagonal shape.

With further examples the cross-sectional geometry of the coding portion of a first coding may distinguish from a second coding or from other codings not only by the cross-sectional geometry but also by the diameter or cross-sectional size. A first coding may comprise a circular shape with a first outer or inner diameter and a second coding may comprise a circular cross-sectional geometry with a different outer or inner diameter, respectively. The same may apply to all other geometric shapes as mentioned above.

By varying the cross-sectional geometry of the coding portion and/or of a longitudinal end face of the insert a large variety of codings can be provided that serves to prevent insertion of the insert of one of the first and second housing components into the receptacle of the other one of the first and second housing components. Variations of the cross-sectional geometries are typically selected such, that each one of the codings can only be inserted into one of a number of available counter codings; and vice versa. Hence, a coding portion of a circular shape cannot be inserted in longitudinal direction into a counter coding portion of oval shape, triangular shape, rectangular shaped or polygonal shape; and vice versa.

According to a further example the mechanical counter coding is defined by a cross sectional geometry of a counter coding portion of a sidewall of the receptacle. With some examples, the entirety of the inside surface of the sidewall of the receptacle may be provided with the mechanical counter coding. With other examples only a portion of the sidewall of the receptacle, e.g. a longitudinal portion of the inside sidewall being located at a predefined longitudinal distance from an insert opening of the receptacle is provided with the mechanical counter coding. Here, the mechanical counter coding may not be directly visible from outside the housing. The coding feature may be thus concealed and may not be visible when the first and second housing components are mutually assembled in a final assembly configuration.

Typically, only an inner cross-sectional geometry of the counter coding portion of the sidewall of the receptacle may be relevant for the coded engagement between the insert and the receptacle. Typically, numerous samples of differently shaped counter coding portions all comprise a sidewall of the receptacle that has a common outer appearance.

According to a further example the cross-sectional geometry of the counter coding portion comprises one of a circular shape, an oval shape, a triangular shape, a rectangular shape and a polygonal shape. As described above in connection with the coding portion of the insert the cross-sectional geometry of the counter coding portion may be adapted accordingly. In this way and when the coding portion comprises a cross-sectional geometry of circular shape and when the counter coding portion comprises a cross-sectional geometry with a corresponding circular shape the insert is insertable into the receptacle. Otherwise, and when the inside cross-sectional geometry of the counter coding portion of the receptacle does not match an outside cross-sectional geometry of the insert the insert is effectively hindered to enter or to completely enter the receptacle along the longitudinal direction.

With some examples and when the counter coding portion is only provided at a predefined offset from an insert opening of the receptacle the insert can be at least partially inserted into the receptacle until the longitudinal end face of the insert gets in axial abutment with the non-matching cross-sectional geometry of the counter coding portion of the inside of the sidewall of the receptacle. Due to this axial abutment, the insert is hindered to completely enter the receptacle. At the same time and when the longitudinal end face of the insert gets in axial abutment, e.g. with a radially stepped down portion of the counter coding as provided on the inside surface of the sidewall of the receptacle, a well-defined and haptic feedback is provided to the user that the coding of the insert does not match with the counter coding of the receptacle.

According to another example the receptacle comprises an inner sidewall portion coaxial with the counter coding portion and forming a circumferential, longitudinally extending counter coding slot with the counter coding portion. The inner sidewall forms or constitutes a supplemental sidewall extending parallel to the sidewall of the receptacle. The inner sidewall may be located inside the receptacle. An axial end of the inner sidewall portion is located entirely inside the receptacle. Typically, a longitudinal end of the inner sidewall portion facing towards the insert opening of the receptacle is located at a predefined longitudinal offset from the insert opening of the receptacle. With some examples the inner sidewall portion is non-visible or hardly visible from outside the housing. It may be located in a longitudinally recessed portion of the receptacle.

The outside surface of the sidewall portion may adapt or conform an inside surface of the counter coding portion of the sidewall of the insert. With some examples, the outside surface of the inner sidewall portion differs from the inside surface of the counter coding portion of the sidewall of the insert. In either way, the counter coding slot is radially outwardly confined by the inner sidewall portion and by the coding portion of the sidewall of the insert. The counter coding slot is radially inwardly confined by an outside surface of the inner sidewall portion. With some examples the longitudinal extent of the inner sidewall portion substantially equals a longitudinal extent of the counter coding portion of the sidewall of the receptacle. A longitudinal end or both longitudinal ends of the inner sidewall portion may coincide with a longitudinal end or with both oppositely located longitudinal ends of the counter coding portion.

The counter coding slot may comprise a homogeneous and non-varying cross-section as seen in longitudinal direction. The same may apply to the coding portion of the insert. This allows and supports a rather smooth and easy insertion of the insert into the counter coding slot.

The counter coding slot comprises a cross-sectional shape that matches the cross-sectional geometry or cross-sectional shape of the coding portion of the sidewall of the insert. When correctly assembled and when an insert with a mechanical coding is inserted into a receptacle featuring a matching mechanical counter coding the coding portion of the sidewall of the insert is located inside the counter coding slot. The counter coding slot is radially outwardly confined by an inside surface of the sidewall of the insert. The counter coding slot is radially inwardly confined by an outside surface of the inner sidewall portion.

The receptacle may be longitudinally confined by an end face extending across an inside diameter of the sidewall of the receptacle. The end face may comprise a radially or transversely extending web, typically comprising a through opening, through which a component of a drive mechanism of the drug delivery device, e.g. the piston rod, may extend. With some examples the inner sidewall portion may be integrally formed or may be connected to the end face that confines the receptacle along the insert direction along which the insert is longitudinally insertable into the receptacle.

With some examples the end face, e.g. in form of an inner end face, is integrally formed with the sidewall of the receptacle. With further examples the inner sidewall portion is integrally formed with the web or end face. The inner sidewall portion may provide increased stability and stiffness to the receptacle. Moreover, the inner sidewall portion and in particular a longitudinal end face of the inner sidewall portion featuring a cross-sectional geometry that matches with the cross-sectional geometry of the mechanical counter coding enhances robustness and stability of the helical counter coding as provided inside the receptacle.

According to a further example the receptacle comprises an insert opening with a first cross section. The counter coding portion is located longitudinally offset from the insert opening and comprises a second cross section. Here, the second cross section is smaller than the first cross section. An inside surface of the sidewall extending longitudinally from the insert opening towards the counter coding portion comprises a beveled surface section. The beveled surface section features a narrowing cross-section or narrowing diameter as seen from the insert opening towards the counter coding portion or towards the inner end face. Insofar the beveled surface section serves to transversely or radially guide the insert into the counter coding portion as provided on the inside of the sidewall of the receptacle. In this way there can be provided a rather smooth and easy insert motion of the insert into the receptacle.

The beveled surface section may be exclusively provided on an inside surface of the sidewall of the receptacle whereas the outside surface of the sidewall of the receptacle is of rather straight or even shape as seen in longitudinal direction. In this way, the thickness of the sidewall of the receptacle may smoothly or abruptly increase in longitudinal direction from the insert opening towards the counter coding portion. In this way, the sidewall of the receptacle can be mechanically stiffened just in the region of the counter coding portion whereas a longitudinal end of the sidewall of the receptacle, e.g. in the region of the insert opening can be provided with a comparatively thin sidewall. Here, the insert opening may exhibit an increased degree of mechanical flexibility compared to the counter coding portion located longitudinally offset from the insert opening. Hence, with some examples, the thickness of the sidewall of the receptacle in a longitudinal section offset from the insert opening is larger than the thickness of the sidewall at the insert opening.

According to another example the mechanical coding comprises at least one of a coding recess and a coding protrusion extending in longitudinal direction. Here, the mechanical counter coding comprises at least one of a counter coding recess matching with the coding protrusion and a counter coding protrusion matching with the coding recess. This type of mechanical coding may be combined with the above-mentioned type of mechanical coding in form of varying cross-sectional geometries of the sidewall of the insert or of the longitudinal end face of the insert. With some examples the coding recess and the coding protrusion and hence the counter coding recess and the counter coding protrusion may be provided as an alternative to the above-mentioned variation of the cross-sectional geometry of the insert and/or of the sidewall of the receptacle. With some examples a longitudinal extent of the coding recess and/or of the coding protrusion provide a mechanical coding to the insert. With other examples, the shape of the coding recess and the coding protrusion define a respective mechanical coding. With still other examples the total number and/or spatial distribution of coding recesses and coding protrusions defines the mechanical coding. With other examples it is the circumferential position of the coding recess and/or of the coding protrusions that defines a respective mechanical coding.

With all these examples the counter coding comprises a correspondingly shaped counter coding feature matching with the coding protrusion and/or matching with the coding recess.

Just as an example the longitudinal end of the first housing component, typically the first connecting end, may comprise a coding recess, e.g. in form of an elongated slit adjoining the longitudinal end face of the first connecting end. Typically, such a recess may be provided on the respective longitudinal end of the insert. The correspondingly shaped counter coding protrusion may be then provided inside the receptacle. It may protrude radially inwardly from the sidewall at a well-defined circumferential position and may comprise a respective longitudinal extent that matches with the longitudinal extent of the coding recess of the insert. The counter coding protrusion may longitudinally extend or protrude from the inner end face of the receptacle.

The coding can be easily varied by modifying the longitudinal extent of the coding recess and/or of the coding protrusion. The coding may be also varied by modifying the number of coding recesses, coding protrusions, counter coding recesses and counter coding protrusion as well as by modifying a tangential or circumferential position as well as a respective geometric shape of coding recesses, coding protrusions, counter coding recesses and counter coding protrusions.

According to a further example the fastening element comprises a snap element. Here, the mechanical coding is defined by at least one of a longitudinal position and a longitudinal extent of the snap element on the insert. With this example the fastening element is integrally formed with the mechanical coding. The mechanical coding and the fastening element may coincide. In other words, the fastening element may provide a double or twofold function. It may provide mutual fastening or fixing of first and second housing components. Additionally, it may prevent a pairing or a mutual assembly of non-matching first and second housing components.

According to a further example the mechanical coding is defined by a relation between the longitudinal position versus the longitudinal extent of the snap element on the insert. Different mechanical codings may distinguish by a different longitudinal position of the snap element on the insert. Here, snap elements located at different longitudinal positions may not only distinguish by their longitudinal position on the insert but also by their longitudinal extent. In this way it can be effectively avoided, that a snap element engages a non-matching counter snap element when spatially overlapping with such a non-matching counter snap element.

Hence, the mechanical coding is encoded by at least two individual parameters, namely by the variation of the longitudinal position on the insert and by the variation of the longitudinal extent of the snap element.

According to a further example the mechanical coding is defined by at least one of a circumferential position and a circumferential extent of the snap element on the insert. Also here, not only the circumferential position of the snap element defines the mechanical coding but also the circumferential extent of the snap element contributes to the mechanical coding. The mechanical coding may be provided by two individual parameters, namely by the circumferential position of the snap element on the insert, e.g. relative to at least one of the groove and the protrusion provided on the insert, and by the circumferential extent on the insert. With some further examples the mechanical coding may be even encoded by at least three individual parameters, namely by the longitudinal position, by the longitudinal extent and by the circumferential extent.

With some further examples the mechanical coding may be defined by four individual parameters, namely by the longitudinal position of the snap element, by the longitudinal extent of the snap element, by the circumferential position of the snap element and by the circumferential extent of the snap element.

Moreover, and according to another example the mechanical coding is defined by a relation between the longitudinal position versus the circumferential extent of the snap element on the insert.

With some examples at least one of the snap element and the counter snap element comprises a radial recess, wherein the other one of the snap element and the counter snap element comprises a correspondingly shaped radial projection to engage with the radial recess. At least one of the radial projection and the radial recess is flexible or elastically deformable, e.g. in radial direction so as to enable a snap fit engagement of the snap element with the counter snap element.

According to a further example the coding feature and the counter coding feature distinguish from coding features and counter coding features of another housing by varying the longitudinal position of a radial recess and the longitudinal position of a radial projection correspondingly, wherein an increase of a longitudinal distance of the radial recess from a free end of the first or second connecting end is accompanied by or combined with an increase of the longitudinal extent and/or by an increase of the transverse or circumferential extent of the radial recess and by a corresponding increase of the longitudinal extent and/or transverse extent of the corresponding radial projection.

According to a further example the counter fastening element comprises a counter snap element. The mechanical counter coding is defined by at least one of a longitudinal position and/or longitudinal extent of the counter snap element in the receptacle.

According to a further example the mechanical counter coding is defined by a relation between the longitudinal position versus the longitudinal extent of the counter snap element inside the receptacle. Different mechanical counter codings may distinguish by a different longitudinal position of the counter snap element in the receptacle. Here, counter snap elements located at different longitudinal positions may not only distinguish by their longitudinal position in the receptacle but also by their longitudinal extent. In this way it can be effectively avoided, that a counter snap element engages a non-matching snap element when spatially overlapping with such a non-matching snap element.

Hence, the mechanical counter coding is encoded by at least two individual parameters, namely by the variation of the longitudinal position in the receptacle and by the variation of the longitudinal extent of the counter snap element.

According to a further example the mechanical counter coding is defined by at least one of a circumferential position and a circumferential extent of the counter snap element in the receptacle. Also here, not only the circumferential position of the counter snap element defines the mechanical counter coding but also the circumferential extent of the counter snap element may contribute or define the mechanical counter coding. The mechanical counter coding may be provided by two individual parameters, namely by the circumferential position of the counter snap element in the receptacle, e.g. relative to at least one of the groove and the protrusion provided in the receptacle. With some further examples the mechanical counter coding may be even encoded by at least three individual parameters, namely by the longitudinal position, by the longitudinal extent and by the circumferential extent.

With some further examples the mechanical counter coding may be defined by four individual parameters, namely by the longitudinal position of the counter snap element, by the longitudinal extent of the counter snap element, by the circumferential position of the counter snap element and by the circumferential extent of the counter snap element.

Moreover, and according to another example the mechanical counter coding is defined by a relation between the longitudinal position versus the circumferential extent of the counter snap element in the receptacle.

According to a further example the coding feature of the first mechanical coding distinguishes from a coding feature of another mechanical coding with regard to at least one of a number of coding features, a longitudinal position, a longitudinal extent, a circumferential position, a circumferential extent and/or by a cross-sectional geometry or shape in a plane transverse to the longitudinal direction. Likewise, and according to further examples the counter coding feature of a first mechanical counter coding distinguishes from a counter coding feature of another mechanical counter coding with regard to at least one of a number of coding features, a longitudinal position, a longitudinal extent, a circumferential position, a circumferential extent and/or a cross-sectional geometry or shape in a plane transverse to the longitudinal direction.

According to a further example the receptacle comprises another counter fastening element located diametrically opposite to the counter fastening element. With further examples, the sidewall of the receptacle is elastically deformable to increase a radial distance between the counter fastening element and the another counter fastening element to such an extent that is larger than or equal a radial distance between the fastening element and another fastening element, which is located diametrically opposite on the insert. In this way and by providing an elastically deformable sidewall of the receptacle the radial distance between diametrically oppositely located counter fastening elements can be increased to such an extent or degree that they disengage from corresponding or complementary shaped fastening elements provided on the insert.

Accordingly, the fastening elements may releasably engage with correspondingly or complementary shaped counter fastening elements. The first and second housing components can be disconnected on demand.

In order to enable or to provide a sufficient elastic deformability of the sidewall of the receptacle it is of particular benefit, when the sidewall of the receptacle at least in the region of its insert opening comprises a comparatively thin side wall. Typically, the sidewall and hence the receptacle and/or the entirety of the respective housing component is made of a plastic material, e.g. an injection molded plastic material inherently providing a sufficient degree of elasticity.

According to a further example the sidewall of the receptacle comprises an outside surface. The outside surface comprises a first flat section and a second flat section radially opposite, e.g. diametrically opposite to the first flat section. A first imaginary straight line intersecting the first flat section and the second flat section extends substantially perpendicular to a second imaginary straight line intersecting the fastening element and the another fastening element. The receptacle and/or the sidewall confining the receptacle may comprise one of a tubular shape, a circular shape and an oval shape.

The first and second flat sections are configured as engaging sections for a particular squeezing tool by way of which the radial distance between the first and second flat sections can be decreased to a predefined degree. A decrease of the radial distance between the first and second flat sections inherently leads to an increase of the radial distance between the first fastening element and the another fastening element. Hence, by applying a radially inwardly directed pressure in opposite directions onto the first and second flat sections the receptacle may adapt or conform a somewhat oval shape, wherein the long axis of the oval shape coincides with the second imaginary straight line intersecting the fastening element and the another fastening element.

The flat section may not only provide a well-defined gripping and squeezing of the housing component but may also provide a visual and haptic guidance for using the squeezing tool.

According to another aspect there is provided an injection device for injecting a dose of a medicament. The injection device comprises a housing as described above and a cartridge arranged inside the housing. The cartridge comprises a barrel filled with a medicament and sealed in a proximal longitudinal direction by a movable bung. The injection device further comprises a drive mechanism arranged inside the housing. The drive mechanism comprises a piston rod operable to exert a distally directed dispensing force onto the bung of the cartridge. Typically, the injection device is implemented as a hand held or portable injection device. The injection device may comprise a pen-type injector.

With some examples the receptacle is provided as a housing insert fixedly attachable or fixedly attached to an elongated housing component, e.g. the first or second housing component of the housing of the drug delivery device. The housing insert may be rotationally and/or longitudinally fixed to the elongated housing component. Insofar all features and benefits as described above in connection with the receptacle equally apply to a housing insert fixedly connectable or fixedly connected to a respective housing component.

According to another aspect the present disclosure relates to a kit of at least a first housing as described above and at least a second housing as described above. Here, the coding feature of the first housing distinguishes from the coding feature of the second housing with regard to at least one of a number of coding features, a longitudinal position, a longitudinal extent, a circumferential position, a circumferential extent and/or a cross-sectional geometry or shape in a plane transverse to the longitudinal direction. Likewise, also the first housing comprises a first counter coding feature that distinguishes from a respective counter coding feature of the second housing with regard to at least one of the above-mentioned features, a longitudinal position, a longitudinal extent, a circumferential position, a circumferential extent and/or a cross-sectional geometry or shape in a plane transverse to the longitudinal direction.

Here, only the housing components of the first housing equipped with complementary shaped mechanical codings and mechanical counter codings are allowed and supported to become mutually fastened and fixed. The first housing is provided with a pair of a coding and counter coding of a first type. A second housing is provided with a pair of a coding and counter coding of a second type. A coding of the first type is incompatible with a counter coding of the second type. A counter coding of the first type is incompatible with a coding of the second type. A user trying to assemble a housing component of the first housing with a housing component of the second housing will be hindered to do so by not-matching mechanical codings and mechanical counter codings.

Generally, and with some examples the first housing components of different housings may distinguish by the size and/or geometry of an accommodating space for receiving a medicament container or cartridge. In particular, a housing with a coding of a first type may be exclusively equipped with a first cartridge or medicament container. A housing with a coding of a second type may be exclusively equipped with a cartridge or a second medicament container. For this, medicament containers, cartridges as well as the interior of the first housing components may comprise further codings or coding features or may distinguish with regard to their size or geometry such that only one dedicated cartridge or medicament container unequivocally fits into only one dedicated first housing component.

With some examples, the first housing component is provided with a mechanical coding to engage with a complementary shaped counter coding of a cartridge. With further examples the first housing component may be provided with at least one of an electronic, a visual or optical coding configured to match with a complementary counter coding of the cartridge, which is also of electronic, visual or optical type.

Moreover, at least one of the cartridge and the first housing component may be provided with a locking or fastening feature by way of which a cartridge can be fixed and/or retained in the first housing component. Here, the first housing component, e.g. implemented as a cartridge holder, and a cartridge assembled therein can be provided as a pre-fabricated housing assembly or as a dedicated cartridge-cartridge holder combination.

In either way, it can be assured or provided that a particular medicament provided in a particular cartridge is unequivocally associated with a particular type of a first housing component, i.e. with a particularly mechanically encoded first housing component. In effect and with some examples, a cartridge provided with a particular medicament can be only accommodated in a correspondingly shaped first housing component equipped with a respective mechanical coding.

With further examples a pre-fabricated housing assembly or a dedicated cartridge-cartridge holder combination is commercially distributed by a pharmaceutical manufacturer. Here, the cartridge may be undetachably or irremovably fixed inside the first housing component and the pharmaceutical manufacturer provides a respective matching between a cartridge filled with a particular medicament and a suitable first housing component, which is mechanically encoded in accordance to the type of medicament located inside the cartridge.

According to a further aspect the present disclosure also relates to a kit of injection devices. The kit of injection devices comprises at least a first injection device comprising a first housing provided with a coding and a counter coding both of a first type and further comprises a second injection device with a second housing provided and equipped with a coding and a counter coding both of a second type non-matching with the respective counter coding or coding of the first type.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE FIGURES

In the following, numerous examples of injection devices with dedicated or coded housing components will be described in greater detail by making reference to the drawings, in which:

FIG. 1 schematically illustrates an example of a drug delivery device,

FIG. 2 shows an example of an exploded view of the drug delivery device of FIG. 1,

FIG. 3 shows an example of first and second housing components of the injection device,

FIG. 4 shows an isolated perspective view of the first housing component,

FIG. 5 shows an enlarged view of a distal end of the second housing component,

FIG. 6 shows another example of a first housing component,

FIG. 7 shows a distal end of another example of a second housing component,

FIG. 8 shows a perspective cross-sectional view of first and second housing components of FIGS. 6 and 7 before assembly,

FIG. 9 shows the housing components of FIG. 8 when reaching a final assembly configuration,

FIG. 10 is another perspective view of the cross-section of the second housing component of FIG. 8,

FIG. 11 shows an example of a counter coding slot of the second housing component,

FIG. 12 shows another example of a counter coding slot of the second housing component,

FIG. 13 shows another example of a counter coding slot of the second housing component,

FIG. 14 shows a further example of a mechanical coding provided on the insert of the first housing component,

FIG. 15 shows a perspective view of the receptacle of the second housing component provided with a respective counter coding,

FIG. 16 is an enlarged view of FIG. 15,

FIG. 17 shows a further example of the mechanical counter coding of FIGS. 15 and 16,

FIG. 18 shows another coding provided on the insert of the first housing component,

FIG. 19 shows another counter coding provided in the receptacle and matching with the mechanical coding of the insert according to FIG. 18,

FIG. 20 shows the transverse deformation capability of the second housing component,

FIG. 21 is a perspective illustration of first and second housing components mutually assembled,

FIG. 22 shows another example of a coding and counter coding as provided on the insert and in the receptacle,

FIG. 23 shows a further example of a coding and a counter coding,

FIG. 24 shows another example of a coding and a counter coding provided on the insert and in the receptacle of first and second housing components,

FIG. 25 is an enlarged perspective view of an insert of a first housing component provided with a radial protrusion,

FIG. 26 is a longitudinal cross-section through mutually assembled first and second housing components,

FIG. 27 is illustrative of an example of a coding and a matching counter coding,

FIG. 28 is illustrative of a further example of a pair of a coding matching with a counter coding,

FIG. 29 is a further example of a mechanical coding matching with a complementary shape mechanical counter coding,

FIG. 30 shows another example of a mechanical coding matching with a mechanical counter coding,

FIG. 31 shows another example of a mechanical coding matching with a mechanical counter coding,

FIG. 32 shows another example of a mechanical coding matching with a mechanical counter coding,

FIG. 33 shows three examples of mechanical codings that distinguish by their cross-sectional geometry as seen in a plane transverse to the longitudinal direction,

FIG. 34 geometrically illustrates one of the mechanical codings of FIG. 33 when implemented on the insert,

FIG. 35 schematically illustrates another of the mechanical codings of FIG. 33 when implemented on the insert, and

FIG. 36 shows another example of the mechanical codings of FIG. 33 when implemented on the insert.

### DETAILED DESCRIPTION

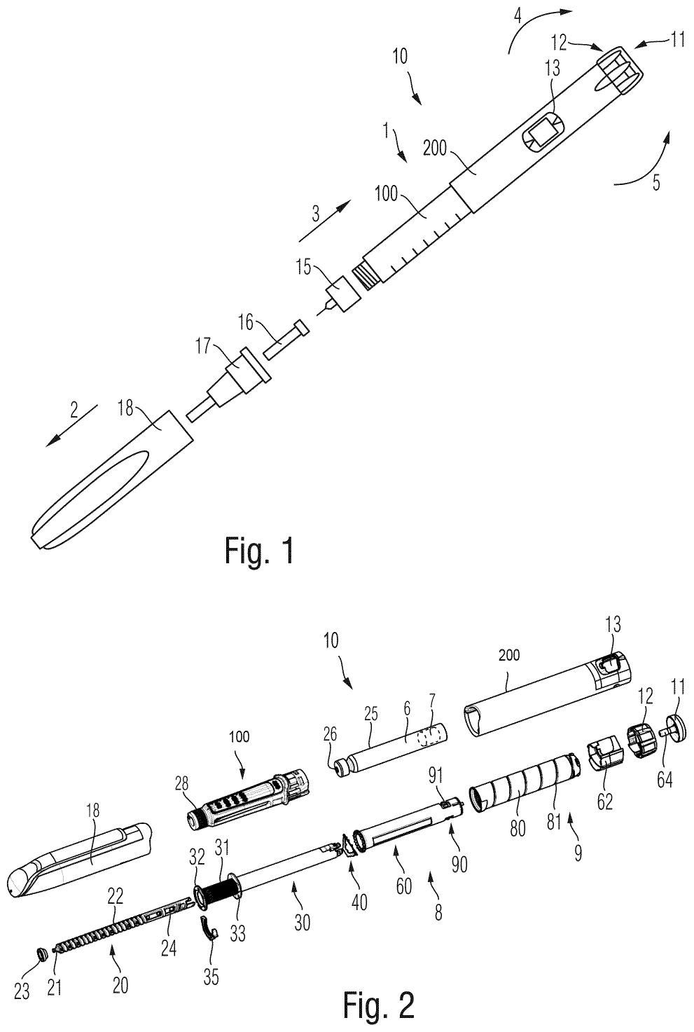

In FIGS. 1 and 2 only one of numerous examples of a handheld injection device is illustrated, that is generally usable in combination with a wearable electronic device. The device as shown in FIGS. 1 and 2 is a pre-filled disposable injection device that comprises a housing 10 to which an injection needle 15 can be affixed. The injection needle 15 is protected by an inner needle cap 16 and either an outer needle cap 17 or a protective cap 18 that is configured to enclose and to protect a distal section of the housing 10 of the injection device 1. The housing 10 comprises a first housing component 100 and a second housing component 200. The second housing component may form a main housing part configured to accommodate a drive mechanism 8 and/or a dose setting mechanism 9 as shown in FIG. 2. The first housing component 100 is configured as a cartridge holder. It may be permanently or releasably connected to the second housing component 200.

The first housing component 100 is typically configured to accommodate a cartridge 6 that is filled with a liquid medicament. The cartridge 6 comprises a cylindrically-shaped or tubular-shaped barrel 25 sealed in proximal direction 3 by means of a bung 7 located inside the barrel 25. The bung 7 is displaceable relative to the barrel 25 of the cartridge 6 in a distal direction 2 by means of a piston rod 20. A distal end of the cartridge 6 is sealed by a pierceable seal 26 configured as a septum and being pierceable by a proximally directed tipped end of the injection needle 15. The cartridge holder and hence the first housing component 100 comprises a threaded socket 28 at its distal end to threadedly engage with a correspondingly threaded portion of the injection needle 15. By attaching the injection needle 15 to the distal end of the first housing component 100 the seal 26 of the cartridge 6 is penetrated thereby establishing a fluid transferring access to the interior of the cartridge 6.

When the injection device 1 is configured to administer e.g. human insulin, the dosage set by a dose dial 12 at a proximal end of the injection device 1 may be displayed in so-called international units (IU, wherein 1 IU is the biological equivalent of about 45.5 μg of pure crystalline insulin (1/22 mg). The dose dial 12 may comprise or may form a dose dial.

As shown further in FIGS. 1 and 2, the housing 10, e.g. the second housing component 200 comprises a dosage window 13 that may be in the form of an aperture in the housing 10. The dosage window 13 permits a user to view a limited portion of a number sleeve 80 that is configured to move when the dose dial 12 is turned, to provide a visual indication of a currently set dose. The dose dial 12 is rotated on a helical path with respect to the housing 10 when turned during setting and/or dispensing or expelling of a dose.

The injection device 1 may be configured so that turning the dosage knob 12 causes a mechanical click sound to provide acoustical feedback to a user. The click sound is typically generated by a click noise generator 45. Generally, a click noise generator 45 may be implemented in various different ways. The number sleeve 80 mechanically interacts with a piston in the insulin cartridge 6. When the needle 15 is stuck into a skin portion of a patient, and when the trigger 11 or injection button is pushed, the dose displayed in display window 13 will be ejected from injection device 1. When the needle 15 of the injection device 1 remains for a certain time in the skin portion after the trigger 11 is pushed, the dose is actually injected into the patient's body. Ejection of a dose of the liquid medicament may also cause a mechanical click sound, which is however different from the click sound produced when using the dose dial 12. For this, the injection device one may comprise a separate, hence a second click noise generator (not illustrated).

In this embodiment, during delivery of the insulin dose, the dose dial 12 is turned to its initial position in an axial movement, that is to say without rotation, while the number sleeve 80 is rotated to return to its initial position, e.g. to display a dose of zero units.

The injection device 1 may be used for several injection processes until either the cartridge 6 is empty or the expiration date of the medicament in the injection device 1 (e.g. 28 days after the first use) is reached.

An example of the drive mechanism 8 is illustrated in more detail in FIG. 2. It comprises numerous mechanically interacting components. A flange like support of the housing 10 comprises a threaded axial through opening threadedly engaged with a first thread or distal thread 22 of the piston rod 20. The distal end of the piston rod 20 comprises a bearing 21 on which a pressure foot 23 is free to rotate with the longitudinal axis of the piston rod 20 as an axis of rotation. The pressure foot 23 is configured to axially abut against a proximally facing thrust receiving face of the bung 7 of the cartridge 6. During a dispensing action the piston rod 20 rotates relative to the housing 10 thereby experiencing a distally directed advancing motion relative to the housing 10 and hence relative to the barrel 25 of the cartridge 6. As a consequence, the bung 7 of the cartridge 6 is displaced in distal direction 2 by a well-defined distance due to the threaded engagement of the piston rod 20 with the housing 10.

The piston rod 20 is further provided with a second thread 24 at its proximal end. The distal thread 22 and the proximal thread 24 are oppositely handed.

There is further provided a drive sleeve 30 having a hollow interior to receive the piston rod 20. The drive sleeve 30 comprises an inner thread threadedly engaged with the proximal thread 24 of the piston rod 20. Moreover, the drive sleeve 30 comprises an outer threaded section 31 at its distal end. The threaded section 31 is axially confined between a distal flange portion 32 and another flange portion 33 located at a predefined axial distance from the distal flange portion 32. Between the two flange portions 32, 33 there is provided a last dose limiter 35 in form of a semi-circular nut having an internal thread mating the threaded section 31 of the drive sleeve 30.

The last dose limiter 35 further comprises a radial recess or protrusion at its outer circumference to engage with a complementary-shaped recess or protrusion at an inside of the sidewall of the housing 10. In this way the last dose limiter 35 is splined to the housing 10, e.g. to the second housing component 200. A rotation of the drive sleeve 30 in a dose incrementing direction 4 or clockwise direction during consecutive dose setting procedures leads to an accumulative axial displacement of the last dose limiter 35 relative to the drive sleeve 30. There is further provided an annular spring 40 that is in axial abutment with a proximally facing surface of the flange portion 33. Moreover, there is provided a tubular-shaped clutch 60. At a first end the clutch 60 is provided with a series of circumferentially directed saw teeth. Towards a second opposite end of the clutch 60 there is located a radially inwardly directed flange.

Furthermore, there is provided a dose dial sleeve also denoted as number sleeve 80. The number sleeve 80 is provided outside of the spring 40 and the clutch 60 and is located radially inward of the housing 10. A helical groove 81 is provided about an outer surface of the number sleeve 80. The housing 10 is provided with the dosage window 13 through which a part of the outer surface of the number sleeve 80 can be seen. The housing 10 is further provided with a helical rib at an inside sidewall portion of an insert piece 62, which helical rib is to be seated in the helical groove 81 of the number sleeve 80. The tubular shaped insert piece 62 is inserted into the proximal end of the housing 10. It is rotationally and axially fixed to the housing 10. There are provided first and second stops on the housing 10 to limit a dose setting procedure during which the number sleeve 80 is rotated in a helical motion relative to the housing 10.

The dose dial 12 in form of a dose dial grip is disposed about an outer surface of the proximal end of the number sleeve 80. An outer diameter of the dose dial 12 typically corresponds to and matches with the outer diameter of the housing 10. The dose dial 12 is secured to the number sleeve 80 to prevent relative movement there between. The dose dial 12 is provided with a central opening.

The trigger 11, also denoted as dose button is substantially T-shaped. It is provided at a proximal end of the injection device 1. A stem 64 of the trigger 11 extends through the opening in the dose dial 12, through an inner diameter of extensions of the drive sleeve 30 and into a receiving recess at the proximal end of the piston rod 20. The stem 64 is retained for limited axial movement in the drive sleeve 30 and against rotation with respect thereto. A head of the trigger 11 is generally circular. The trigger side wall or skirt extends from a periphery of the head and is further adapted to be seated in a proximally accessible annular recess of the dose dial 12.

To dial a dose a user rotates the dose dial 12. With the spring 40, also acting as a click noise generator 45, and the clutch 60 engaged, the drive sleeve 30, the spring 40, the clutch 60 and the number sleeve 80 rotate with the dose dial 12. Audible and tactile feedback of the dose being dialed is provided by the spring 40 and by the clutch 60. Torque is transmitted through saw teeth between the spring 40 and the clutch 60. The helical groove 81 on the number sleeve 80 and a helical groove in the drive sleeve 30 have the same lead. This allows the number sleeve 80 to extend from the housing 10 and the drive sleeve 30 to climb the piston rod 20 at the same rate. At a limit of travel a radial stop on the number sleeve 80 engages either with a first stop or a second stop provided on the housing 10 to prevent further movement in a first sense of rotation, e.g. in a dose incrementing direction 4. Rotation of the piston rod 20 is prevented due to the opposing directions of the overall and driven threads on the piston rod 20.

The last dose limiter 35 keyed to the housing 10 is advanced along the threaded section 31 by the rotation of the drive sleeve 30. When a final dose dispensed position is reached, a radial stop formed on a surface of the last dose limiter 35 abuts a radial stop on the flange portion 33 of the drive sleeve 30, preventing both, the last dose limiter 35 and the drive sleeve 30 from rotating further.

Should a user inadvertently dial beyond the desired dosage, the injection device 1, configured as a pen-injector allows the dosage to be dialed down without dispense of the medicament from the cartridge 6. For this the dose dial 12 is simply counter-rotated. This causes the system to act in reverse. A flexible arm of the spring or clicker 40 then acts as a ratchet preventing the spring 40 from rotating. The torque transmitted through the clutch 60 causes the saw teeth to ride over one another to create the clicks corresponding to dialed dose reduction. Typically, the saw teeth are so disposed that a circumferential extent of each saw tooth corresponds to a unit dose. Here, the clutch may serve as a ratchet mechanism.

As an alternative or in addition the ratchet mechanism 90 may comprise at least one ratchet feature 91, such as a flexible arm on the sidewall of the tubular-shaped clutch 60. The at least one ratchet feature 91 may comprise a radially outwardly extending protrusion e.g. on a free end of the flexible arm. The protrusion is configured to engage with a correspondingly shaped counter ratchet structure on an inside of the number sleeve 80. The inside of the number sleeve 80 may comprise longitudinally shaped grooves or protrusions featuring a saw-tooth profile. During dialing or setting of a dose the ratchet mechanism 90 allows and supports a rotation of the number sleeve 80 relative to the clutch 60 along a second sense of rotation 5, which rotation is accompanied by a regular clicking of the flexible arm of the clutch 60. An angular momentum applied to the number sleeve 80 along the first sense of rotation for is unalterably transferred to the clutch 60. Here, the mutually corresponding ratchet features of the ratchet mechanism 90 provide a torque transmission from the number sleeve 80 to the clutch 60.

When the desired dose has been dialed the user may simply dispense the set dose by depressing the trigger 11. This displaces the clutch 60 axially with respect to the number sleeve 80 causing dog teeth thereof to disengage. However, the clutch 60 remains keyed in rotation to the drive sleeve 30. The number sleeve 80 and the dose dial 12 are now free to rotate in accordance with the helical groove 81.

The axial movement deforms the flexible arm of the spring 40 to ensure the saw teeth cannot be overhauled during dispense. This prevents the drive sleeve 30 from rotating with respect to the housing 10 though it is still free to move axially with respect thereto. The deformation is subsequently used to urge the spring 40 and the clutch 60 back along the drive sleeve 30 to restore the connection between the clutch 60 and the number sleeve 80 when the distally directed dispensing pressure is removed from the trigger 11.

The longitudinal axial movement of the drive sleeve 30 causes the piston rod 20 to rotate through the through opening of the support of the housing 10, thereby to advance the bung 7 in the cartridge 6. Once the dialed dose has been dispensed, the number sleeve 80 is prevented from further rotation by contact of at least one stop extending from the dose dial 12 with at least one corresponding stop of the housing 10. A zero-dose position may be determined by the abutment of one of axially extending edges or stops of the number sleeve 80 with at least one or several corresponding stops of the housing 10.

The expelling mechanism or drive mechanism 8 as described above is only exemplary for one of a plurality of differently configured drive mechanisms that are generally implementable in a disposable pen-injector. The drive mechanism as described above is explained in more detail e.g. in WO2004/078239A1, WO 2004/078240A1 or WO 2004/078241A1 the entirety of which being incorporated herein by reference.

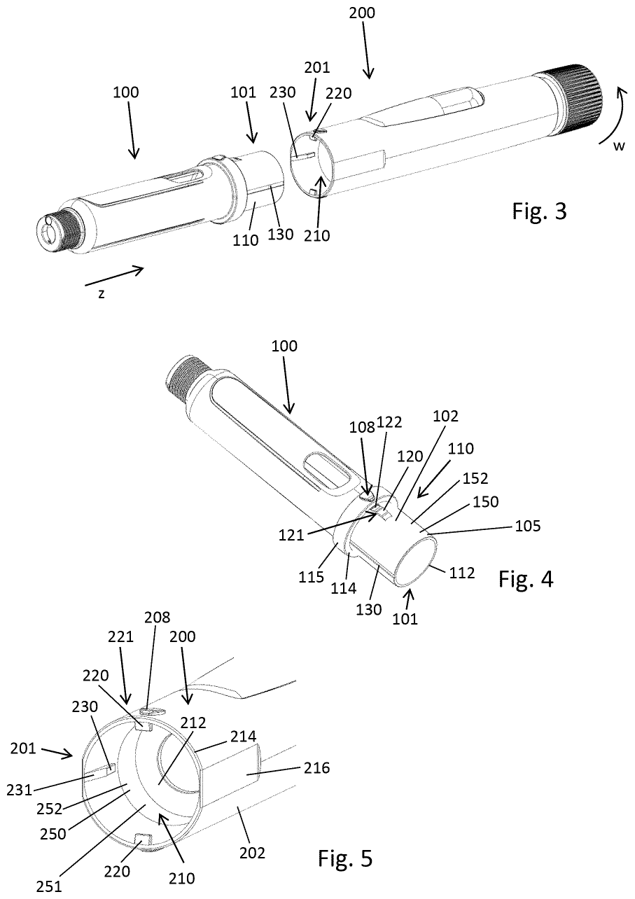

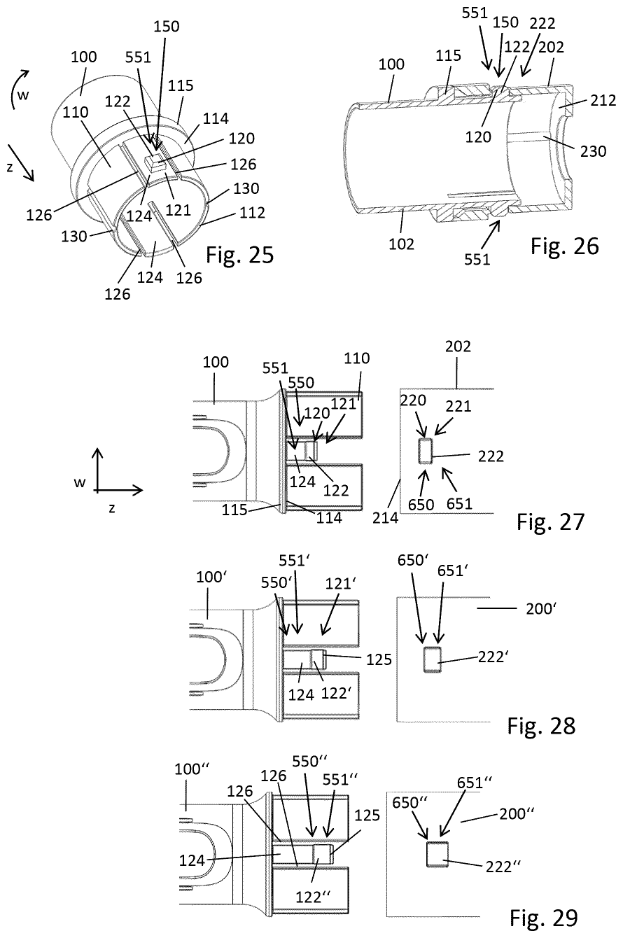

The housing 10 as illustrated in any of the FIGS. 3-36 comprises a first housing component 100 and a second housing component 200. The first housing component 100 is configured as a cartridge holder. It is sized and shaped to accommodate a cartridge 6 inside its hollow interior. The cartridge holder and hence the first housing component 100 comprises a first connecting end 101. The first connecting end 101 forms a proximal end of the first housing component 100. Correspondingly, the second housing components 200 comprises a second connecting end 201, typically at a distal end of the housing component 200.

The first connecting end 101 is mechanically connectable to the second connecting end 201. As illustrated, the first housing component 100 comprises an insert 110 forming the first connecting end 101. The second housing component 200 comprises a receptacle 210 shaped and sized to receive the insert 110. The insert 110 is insertable into the receptacle 210 by a longitudinal sliding movement relative to the second housing component 200, in particular along the proximal direction 3.

The insert 110 forms a proximal end of the first housing component 100. The insert 110 comprises a proximal end face 112. Towards the distal direction 2 the insert 110 is confined by a flange section 115 protruding radially outwardly from the tubular shaped sidewall 102 of the first housing component 100 and hence also from a sidewall 102 of insert 110.

The flange section 115 comprises a circumferential rim extending all around the tubular shaped insert 110. Towards the proximal direction 3 the flange section 115 comprises an abutment face 114 facing in proximal direction 3. The abutment faces 114 is configured to axially abut a distal end face 214 of the sidewall 202 of the second housing component.

For mutually fixing the first and second housing components 100, 200 there is provided a fastening element 120 on the insert 110 to operably engage with a correspondingly or complementary-shaped counter fastening element 220 provided inside the receptacle 210. In the presently illustrated examples, as for instance shown in greater detail in FIGS. 4 and 5 the fastening element 120 comprises a snap element 121 configured to engage with the correspondingly or complementary shaped counter snap element 221 as provided on an inside surface 203 of the sidewall 202 of the receptacle 210. The snap element 121 comprises a radial protrusion 122 as illustrated in FIG. 8 and a radial recess 127 shaped and configured to engage with and/or to receive a complementary shaped radial projection 222 of the counter fastening element 220 protruding radially inwardly from the sidewall 202 of the receptacle 210.

As shown in FIG. 8, there are provided numerous fastening elements 120 and complementary-shaped counter fastening elements 220 on the outside surface of the sidewall 102 of the insert 110 and on the inside surface 203 of the sidewall 202 of the receptacle 210, respectively. By way of mutually corresponding snap elements 121 and counter snap elements 221 a snap-fit engagement of the first and second housing components 100, 200 can be provided.

On the outside surface 105 of the insert 110 there is further provided a longitudinal groove 130 extending from the longitudinal end face 112 to the flange section 115. In the presently illustrated example, there are provided two diametrically oppositely located longitudinal grooves 130. On the inside surface 203 of the receptacle 210 there is provided at least one protrusion 230 that is shaped and sized to engage with and to slide along the groove 130 upon insertion of the insert 110 into the receptacle 210. The protrusion 230 and the groove 130 provide a keyed engagement of the insert 110 and the receptacle 210. By way of the keyed engagement the insert 110 is rotationally locked to the receptacle 210. Thus, a mutual fixing and fastening of the first housing component 100 and the second housing components 200 is obtained by a purely longitudinal sliding motion of the insert 110 into the receptacle 210.

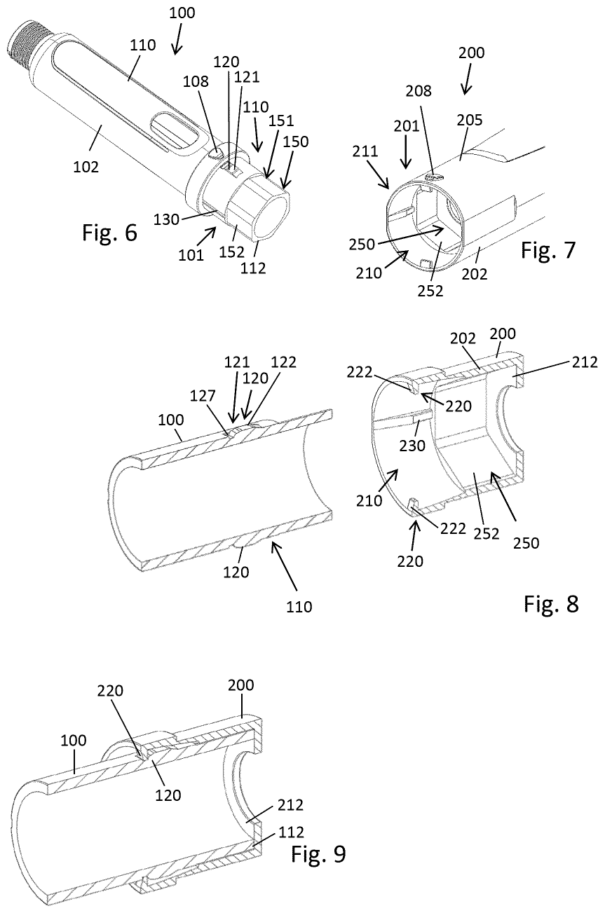

As further illustrated in FIGS. 6 and 7 there is provided an indicator 108 on the outside surface 105 of the first housing component 100. There is provided a complementary or correspondingly shaped indicator 208 on the outside surface 205 of the second housing components 200. When reaching a final assembly configuration, the indicator 108 and the indicator 208 are mutually aligned in longitudinal direction (z). This way, and in the course of inserting the insert 110 into the receptacle 210 the indicator 108 and the indicator 208 provide a visual guiding for the user how to align or to orient the insert 110 relative to the receptacle 210 to support a smooth insertion of the insert 110 into the receptacle 210.

As illustrated in FIG. 8 the receptacle 210 is delimited or confined in proximal direction by an end face 212 protruding radially inwardly from the sidewall 202. The end face 212 may axially abut with the end face 112 upon reaching a final assembly configuration of first and second housing components 100, 200. The radially inwardly protruding end face 212 comprises or forms a radially inwardly protruding flange, e.g. integrally formed with the sidewall 202 of the second housing component 200.

The insert 110 is further provided with a mechanical coding 150. The receptacle 210 is provided with a complementary shaped mechanical counter coding 250. The mechanical coding 150 comprises a coding feature 151. The coding feature 151 may be defined in different ways as will become apparent by the illustration of numerous examples of the mechanical coding 150, 350, 550, 750.

For instance, and in the example of FIGS. 3-13 the mechanical coding 150 is defined by a cross-sectional geometry of a coding portion 152 of the sidewall 102 of the insert 110. In the example of FIG. 3, the cross-sectional geometry of the insert 110 is of circular shape. The correspondingly or complementary shaped counter coding 250 is provided with a respective cross-sectional geometry of circular shape.

In the example of FIGS. 6-9 and FIGS. 11-13 the coding portion 152 of the insert 110 is of polygonal shape. Accordingly, the inner cross-sectional geometry of the complementary shaped counter coding portion 252 as provided on the inside surface 203 of the receptacle 210 also comprises a respective polygonal shape as indicated in FIG. 8. In the sequence of FIGS. 11-13, numerous different geometries of a cross-sectional shape of the counter coding feature 250′, 250″ and 250″ are schematically illustrated.

Generally, the coding portion 152 provided at the outside surface 105 of the insert 110 is correspondingly shaped to a respective counter coding portion 252. With a hexagonal shaped coding 150 and a respective coding feature 151 comprising a hexagonal cross-section at the coding portion 152 as shown in FIG. 11, the cross-sectional geometry of the counter coding 250′ and a respective counter coding feature 251′ of a second type also comprises a matching hexagonal shape. Other types of codings and counter codings may differ in shape and/or geometry. The cross-sectional geometry of the counter coding portion 250″ and its respective counter coding feature 251″ of a third type as shown in FIG. 12 comprises an octagonal shape and the cross-sectional geometry of the counter coding feature 251″ as shown in FIG. 13 comprises 12 corners.

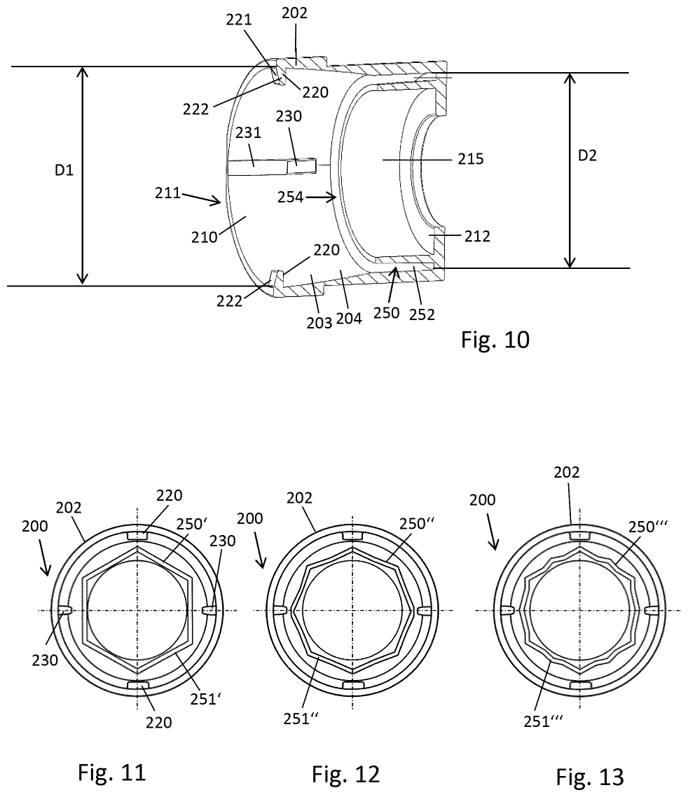

As further illustrated in FIG. 10, the receptacle 210 is not only confined and defined by the inside surface 203 of the sidewall 202 of the second housing components 200. The counter coding 250 and the respective counter coding portion 252 may be further defined by an inner sidewall portion 215 co-axial with the sidewall 202 of the second housing component 200. The inner sidewall portion 250 comprises an outer diameter that is smaller than an inner diameter of the inside surface 203 of the sidewall 202 of the receptacle 210. Hence, there is provided a counter coding slot 254, which is open towards the distal direction 2 and which is shaped and configured to receive the coding portion 152 at the proximal connecting end 101 of the insert 110.

In the example of FIG. 10 the counter coding slot 254 is of circular geometry. In this way, only the coding portion 152 as for instance illustrated in FIG. 4 will be allowed to enter and to slide into the counter coding slot 254 in longitudinal direction (z). Any other available cross-sectional geometry of a coding portion 152, such as a coding portion 152 as illustrated in FIG. 6 will be hindered to enter the counter coding slot 254. In this way, it is effectively prevented that the insert 110 completely enters the receptacle 210. The insert 110 is hindered to arrive in the predefined final assembly configuration, in which the fastening element 120 engages with the counter fastening element 220.

The supplemental inner sidewall portion 215 further enhances the mechanical rigidity and stability of the receptacle 210.

As further illustrated in FIG. 10 the receptacle 210 comprises an insert opening 211. The insert opening 211 is provided at the distal end of the receptacle 210. The insert opening 211 comprises a first cross section and/or a first diameter D1 which is larger than a second cross-section or diameter D2 provided in a longitudinal region located longitudinally offset from the insert opening 211. Accordingly, the inside surface 203 of the sidewall 202 comprises a beveled surface section 204 in the region between the insert opening 211 and the counter coding portion 252.

The beveled surface section 204 radially narrows towards the proximal direction 3. The beveled surface section 204 radially narrows towards and/or along an insert direction into the receptacle 210. In this way, the coding portion 152 and/or the proximal end of the insert 110 is radially guided to smoothly engage with the counter coding portion 252 and/or with the counter coding slot 254.

Additionally, the radially narrowing beveled surface section 204 comes along with the benefit to reduce the radial thickness of the sidewall 202 in the region of the insert opening 211 compared to longitudinal regions of the sidewall 202 located longitudinally offset from the insert opening 211. A reduced thickness of the sidewall 202 towards the insert opening 211 provides the benefit to increase the mechanical elasticity of the sidewall 202 at least in the region of the insert opening 211. In this way, the sidewall 202 of the receptacle 210 can be easily deformed elastically, at least in the longitudinal region of the insert opening 211.

As further illustrated in FIGS. 5 and 10 also the protrusion 230 may comprise a chamfered end section 231 towards the distal end and towards the insert opening 211. In this way, the radial size of the protrusion 230 reduces towards the distal direction. This provides a rather smooth engagement of the protrusion 230 with the elongated groove 130 on the insert 110. Moreover, the size reduction of the protrusion 230 towards the insert opening 211 facilitates and supports an elastic deformation of the sidewall 202 in the region of the insert opening 211, especially when the insert 110 is located inside the receptacle 210. This may support and facilitate an elastic deformation of the sidewall 202 as will be described in connection with FIG. 20, e.g. for the purpose of disconnecting the first and second housing components 100, 200.

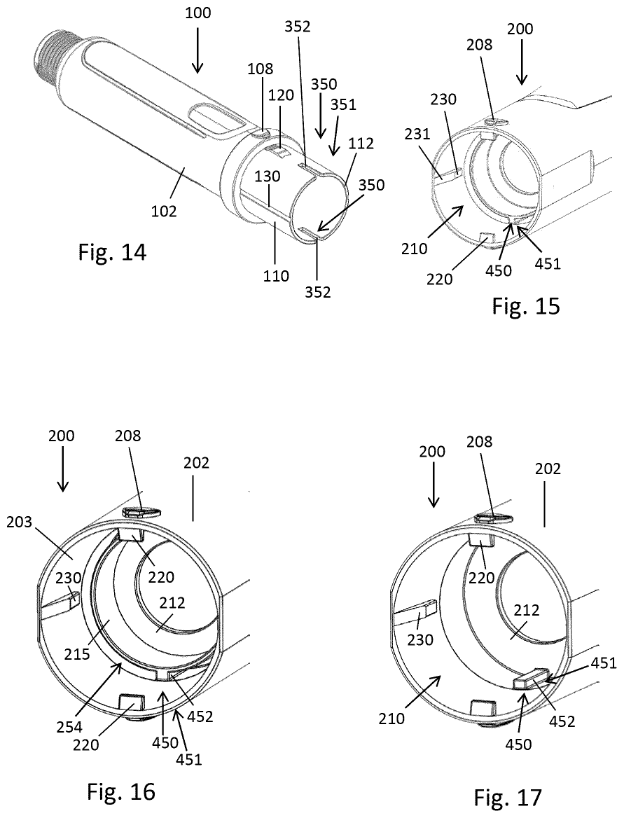

In the example of FIGS. 14-19 another mechanical coding 350 with a coding feature 351 and a complementary shaped counter coding 450 with a counter coding feature 451 is schematically illustrated. Here, the insert 110 comprises a coding recess 352 extending in longitudinal direction (z). The corresponding counter coding 450, hence the counter coding feature 451 comprises a complementary shaped counter coding protrusion 452. The coding feature 351 and the complementary shaped counter coding feature 451 may be defined by the longitudinal extent of the coding recess 352 and by the complementary longitudinal extent of the counter coding protrusion 452.

The first housing component 100 may distinguish from other first housing component 100 by one of a longitudinal extent of the coding recess 352 and a circumferential position or extent of the coding recess 352. The coding recess 352 comprises or forms an elongated slit or slot in the sidewall 102 of the insert 110 adjoining the end face 212.

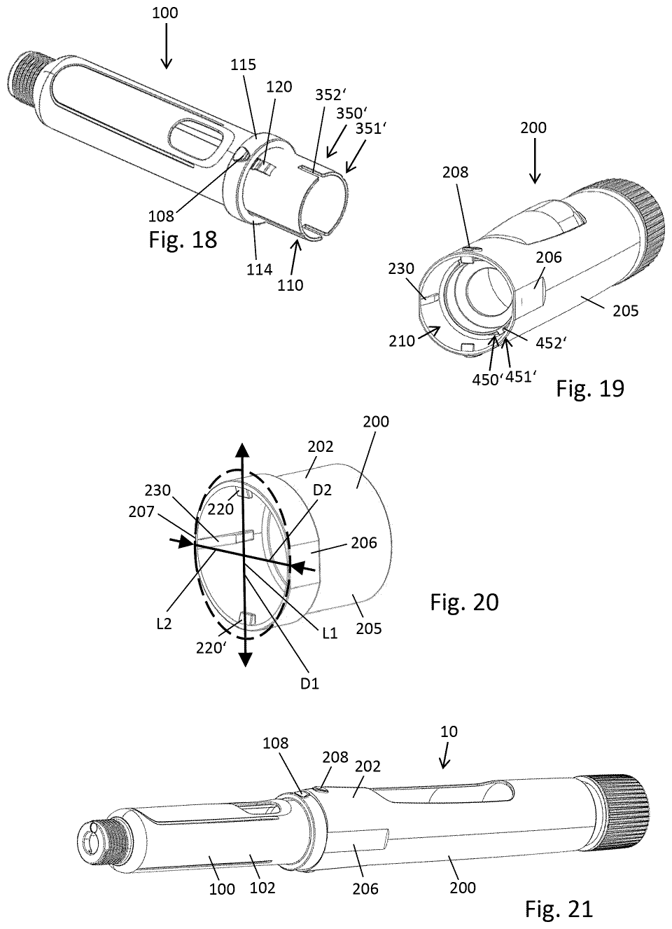

Compared to FIG. 14, the further mechanical coding 350′ of another first housing component 100′ as shown in FIG. 18 distinguishes from the mechanical coding 350 of the housing component 100 of FIG. 14 by varying the circumferential position of the mechanical coding 350′ and the respective mechanical coding feature 351′.

Here, the coding recess 352′ is located at a different circumferential position relative to the groove 130 and/or relative to the fastening element 120 as compared with the coding recess 352 as illustrated in FIG. 14.

Correspondingly and as illustrated in FIG. 19 the second housing components 200 is equipped with a complementary shaped mechanical counter coding 450′. The mechanical counter coding feature 451′ comprises a counter coding protrusion 452′ that distinguishes from the counter coding protrusion 452 as shown in FIG. 17 by its circumferential position. In this way, it is provided that only in insert 110 provided with a mechanical coding 350 can be inserted into a receptacle 210 as shown in FIG. 17, which comprises a complementary shaped counter coding 450. The housing component 100 as shown in FIG. 14 cannot be connected with the housing component 200 as illustrated in FIG. 19 because the mechanical coding 350 does not match with the mechanical counter coding 450′.

Likewise, the insert 110′ of the first housing component 100 as shown in FIG. 18 cannot be inserted into the receptacle 210 of a second housing component 200 of FIG. 15 or FIG. 17 because the circumferential position of the coding feature 350′ does not match with the circumferential position of the mechanical counter coding feature 450.

In FIG. 16 the receptacle 210 is also provided with the inner sidewall portion 215. Here, the protrusion 452 is located radially between the inside surface 203 and an outside surface of the sidewall 202 and the inner sidewall portion 215. Here, the mechanical counter coding protrusion 452 mechanically connects the inner sidewall portion 215 with the outer sidewall 202, thereby increasing the mechanical stability and stiffness of the receptacle 210.

In the illustration of FIG. 20 there are provided two imaginary straight lines L1 and L2. The first imaginary straight line L1 extends through diametrically oppositely located counter fastening elements 220, 220′. The second imaginary straight line L2 extend through a first flat section 206 and a diametrically oppositely located second flat section 207 on the outside surface 205 of the sidewall 202 of the receptacle 210.

The flat sections 206, 207 are located on the outside surface 205 of the sidewall 202, e.g. in the region of or longitudinally adjacent to the insert opening 211, and hence at or near a distal connecting end 201 of the second housing component 200.

The first and second flat sections 206, 207 provide a well-defined engagement with a squeezing tool, such as pliers (not illustrated). By applying a radially inwardly directed pressure onto the oppositely located flat sections 206, 207, the radial distance or cross-section between the flat sections 206, 207 can be reduced, thereby increasing the radial distance between the counter fastening element 220 and the another counter fastening element 220′. Accordingly, the original and somewhat circular cross-section of the insert opening 211 is elastically deformed to adapt a somewhat oval shape.

By increasing the radial distance between the counter fastening element 220 and the diametrically oppositely located counter fastening element 220′, the counter fastening element(s) 220, 220′ may disengage from the complementary shaped fastening element(s) 120, 120′ as provided on the insert 110. In this way, the first and second housing components 100, 200 may disengage and can be disassembled. Disengagement of first and second housing component 100, 200 may allow replacement of an empty cartridge 6 and a further use of the drug delivery device 1 with a new cartridge 6.

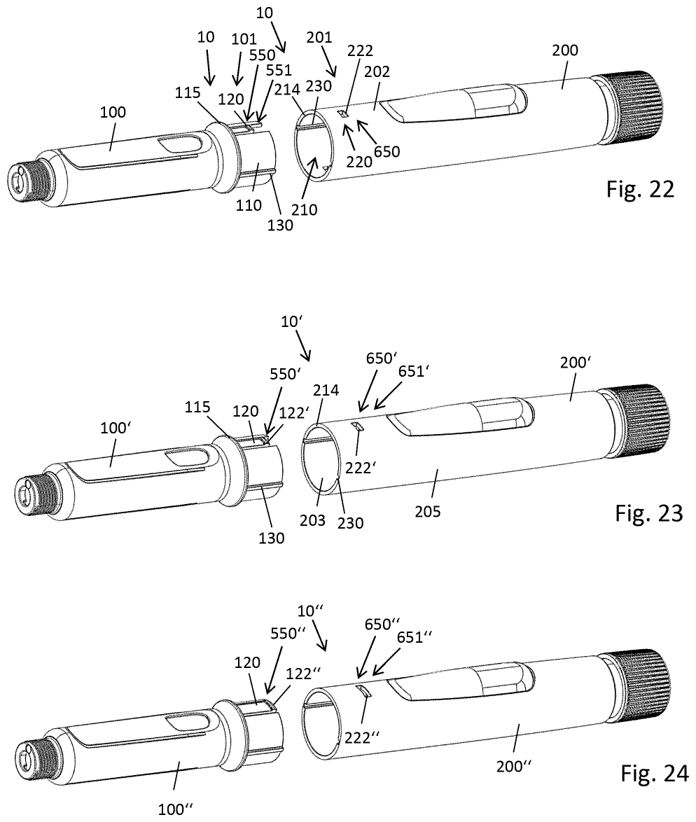

In FIGS. 22-29 another example of a mechanical coding 550 complementary shaped to a mechanical counter coding 650 is schematically illustrated. As shown in greater detail in FIG. 25 the insert 110 of the first housing component 100 comprises a fastening element 120 featuring a snap element 121 configured to engage with a complementary-shaped counter snap element 221 of a counter fastening element 220 as provided on the second housing component 200.

The fastening element 120 is implemented as a snap element 121. It comprises a radial protrusion or projection 122 complementary shaped to a radial recess 222 in the sidewall 202 of the second housing component 200. The radial recess 222 is presently illustrated as a through recess extending entirely through the sidewall 202. With other examples the radial recess 222 is a blind recess only provided on the inside surface of 203 of the receptacle 210. Here, the outside surface 205 of the second housing components 200 is void of any recesses or the like counter fastening elements 220.

In the illustrated example the radial protrusion 122 of the snap element 121 protrudes radially outwardly from the insert 110. It may be elastically deformable in radial direction. This may be achieved by providing the radially outwardly extending radial protrusion 122 on the outside surface of a tongue portion 124. The tongue portion 124 is a part of the insert 110 but is separated from the sidewall of the insert 110 by a first and a second longitudinal slit 126 confining the tongue portion 124 in circumferential direction (w). The slits 126 extend in longitudinal direction (z) from the flange section 115 towards the longitudinal end face 212. The tongue portion 124 may be exclusively connected to the flange section 115. It may be elastically bendable, elastically deformable and/or pivotably supported on the insert 110 with regard to the radial direction.

At a longitudinal end of the tongue portion 124 facing towards the insert direction and hence facing towards the free end of the respective housing component 100 there is provided a beveled section or chamfer 125. This beveled section or chamfer 125 induces and supports a radially inwardly directed bending or flexing of the tongue portion 124 when getting in contact with the sidewall 202 of the receptacle 210.

The fastening element 120 is integrally formed with the mechanical coding 550. In other words, the mechanical coding 550 may be integrated into the fastening element 120; and vice versa. Accordingly, the protrusion 122 forms or constitutes the mechanical coding feature 551. Accordingly, the radial recess 222 as provided in the sidewall 202 of the receptacle 210 defines the mechanical counter coding 650. At least one of the geometry, the geometric extend and/or the position of the radial protrusion 122 defines the mechanical coding feature 551. Likewise, at least one of the geometry, the extent, the size and/or the position of the radial recess 222 defines the mechanical counter coding features 651.

In the example as illustrated in the sequence of FIGS. 27-29 there are illustrated three different pairings of mutually corresponding mechanical coding 550, 550′, 550″ and respective mechanical counter codings 650, 650′, 650″ of a first type, a second type and of a third type, respectively. Insofar, each one of the FIGS. 27-29 illustrates a particularly encoded housing 10, 10′, 10″ of a drug delivery device 1. A first housing component 100 of a housing 10 cannot engage with a second housing component 200′, 200″ of any of the other housings 10′, 10″. Vice versa, a second housing component 200 of the housing 10 cannot engage with a first housing component 100′, 100″ of any one of the other housings 10′, 10″.

Insofar, FIGS. 27-29 are illustrative of a kit of numerous housings and 10, 10′, 10″ of injection devices, wherein a first housing component 100 of a selected housing 10, 10′, 10″ is exclusively connectable to a second housing component 200 of the same housing 10 and is prevented from connecting with a second housing component 200′, 200″ of any of the other housings 10′, 10″ of the kit of housings.

The mechanical coding feature 551 is defined by a combination of the longitudinal position of the fastening element 120 and hence by a combination of the longitudinal position of the respective snap element 121 and the longitudinal extent of the snap element 121 and/or of its radial protrusion 122. Generally, the snap element 121 can be regarded as a coding feature 551 and the radial protrusion 122 can be regarded as a radial coding protrusion 552.

As can be seen in the sequence of FIGS. 27-29 the longitudinal position of the coding feature 551, 551′, 551″ varies with regard to the longitudinal direction (z). In the same way also the longitudinal extent of the coding feature, in particular the longitudinal extent of the radial protrusion 122, 122′, 122″ varies. In the same way also the longitudinal position of the complementary shaped counter coding features 651, 651′, 651″ and the respective longitudinal extent of the radial recess 222, 222′, 222′ varies accordingly. In this way it can be ensured, that the mechanical coding 550 is exclusively pairable, combinable and/or engageable with the mechanical counter coding 650.

In a final assembly configuration, i.e. when the proximal face 114 of the flange section 115 axially abuts with the distal end face 214 of the second housing component 200 the radial protrusion 122 of the snap element 121 engages the radial recess 222 of the complementary shaped counter fastening element 220. As illustrated, the longitudinal extent of the radial protrusion 122 closely matches the longitudinal extent of the radial recess 222. As the longitudinal extent of the radial protrusion 122′, 122″ gradually increases as it is apparent by the examples of FIGS. 28 and 29 the longitudinal position of the respective coding feature 551′, 551″ separates more and more from the abutment face 114.

Accordingly, the longitudinal distance of the radial recess 222 and hence of the counter coding features 651′, 651″ separates more and more from the end face 214.

Accordingly, by varying the longitudinal position of the radial recess 222, 222′, 222″ and the longitudinal position of the radial protrusion 122, 122′, 122″ correspondingly, an increase of a longitudinal distance of the radial recess 222, 222′, 222″ on a free end of the second connecting end 201 is accompanied by an increase of the longitudinal extent of the radial recess 222, 222′, 222″ and by a corresponding increase of the longitudinal extent of the correspondingly shaped radial protrusion 122, 122′, 122″. In this way it is guaranteed, that the coding feature 551 is exclusively engageable with only one particular counter coding feature 651 of the available counter coding features 651, 651′, 651″.

In an attempt that the coding feature 551 would be paired with one of the non-matching or incompatible counter coding features 651′, 651″, hence when attempting to engage the coding feature 551 of the insert 110 of FIG. 27 into the receptacle 210 provided with the counter coding features 651′, 655′ as shown in FIG. 28 or 29 the radial protrusion 122 will not reach the longitudinal position of the respective counter coding features 651″. Before spatially overlapping with any of the counter coding features 651′, 651″ the abutment face 114 engages the axial end face 214 and blocks any further proximally directed movement of the insert 110 into the receptacle 210.

The other way round and when attempting to insert any of the coding feature 551′, 551″ of any of the FIG. 28 or 29 into the receptacle 210 of FIG. 27 the radial protrusion 122′, 122″ may slide over and across the radial recess 222. But since the longitudinal extent of the radial protrusions 122′, 122″ is larger than the longitudinal extent of the radial recess 222 the radial protrusions 122′, 122″ cannot engage the radial recess 222. The longitudinal extent of the recess 222 is smaller than the longitudinal extent of the radial protrusions 122′, 122″. Accordingly, the coding features 551′, 551″ are hindered to engage the counter coding 651. Due to this difference with regard to the longitudinal extent the snap elements 121′, 121″ cannot engage the counter snap element 221.

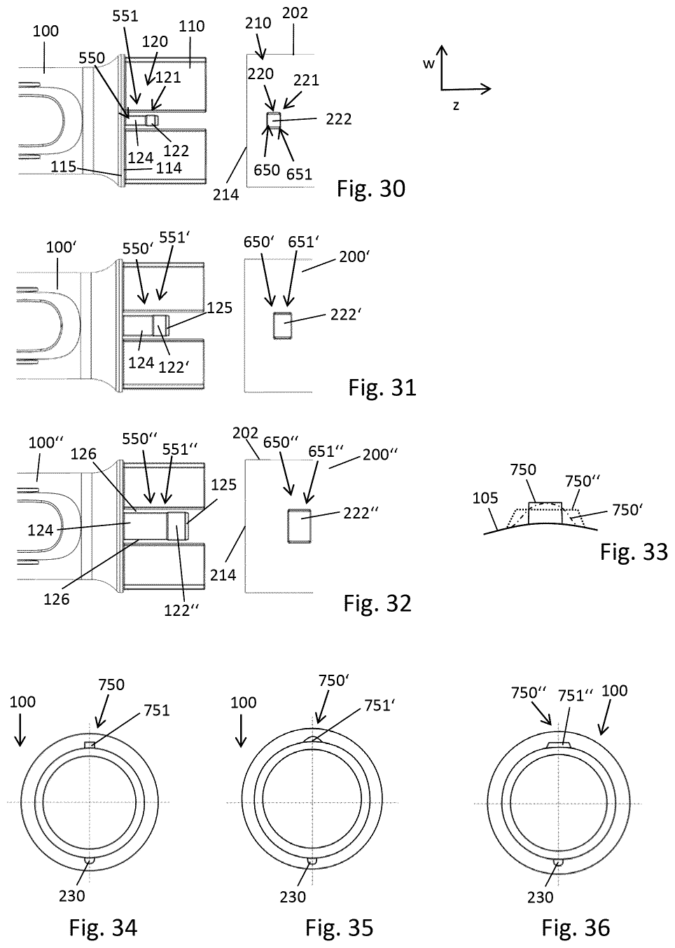

In the further illustration of the sequence of FIGS. 30-32, another example of a mechanical coding 550, 550′, 550″ of a kit of housings 10, 10′, 10″ is illustrated. Here, not only the longitudinal position and/or the longitudinal extent of the snap element 121, 121′, 121″ is subject to a modification and geometric variation but als 121o the extent of the respective snap element 121, 121′, 121″ with regards to the circumferential extend, hence along the circumferential direction (w) is subject to a respective modification.

The size and position of the complementary shaped counter coding features 650, 650′, 650″ and perspective counter coding features 651, 651′, 651″ is subject to a respective modification and variation. As becomes apparent from the illustration of the sequence of FIGS. 30-32 the radial protrusion 122′ is located further away in longitudinal direction from the abutment face 214 than the radial protrusion 122. The same applies to the radial recess 222′ in relation to the radial recess 222 with regards to the longitudinal end face 214. At the same time the longitudinal extent of the radial protrusion 122′ is larger than the longitudinal extent of the radial protrusion 122.

The same applies by a comparison of the radial protrusion 122″ with regards to the radial protrusion 122′. The circumferential extent or size of the radial protrusion 122″ is larger than the circumferential extend or size of the radial protrusion 122′. In this way and when attempting to engage one of the mechanical coding 550′, 550″ or one of the coding features 551′, 551″ with the mechanical counter coding 650 or mechanical counter coding feature 651 the respective radial protrusion 122′, 122″ fails to engage with the counter coding features 651 or radial recess 222 because the circumferential extent of radial recess 222 is smaller than the circumferential extent of any of the radial protrusions 122′, 122″.

In FIG. 33 there are provided three examples of three further mechanical codings 750, 750′, 750″ as they might be provided on an outside surface 105 of a sidewall 102 of the insert 110. The different mechanical codings 750, 750′, 750″ vary with regard to their transverse size, cross-sectional shape and/or geometry. The mechanical coding 750 comprises a somewhat rectangularly shaped mechanical coding feature 751. The mechanical coding 750′ comprises a convex-shaped radially outwardly protruding coding feature 751′ and the further example of a mechanical coding 750″ comprises a trapezoidal-like cross-section or geometry. Any one of the coding features 751, 751′, 751″ distinguishes from the other two of the coding features 751, 751′, 751″ at least with regards to the radial extent and/or circumferential extent. In this way, it can be guaranteed, that each one of the mechanical coding features 751, 751′, 751″ can engage with only one of a complementary-shaped counter coding feature, which are not illustrated in greater detail here.

The mechanical coding 750, 750′, 750″ is at a well-defined circumferential position relative to the radial protrusion 230, which is configured to engage with the groove 130. With the example of FIGS. 34-36, the radial protrusion 230 is provided on the insert 110 and the groove 130 is provided on an inside surface 203 of the receptacle 210.

With the presently illustrated examples the insert 110 is provided on the first housing component 100 and the receptacle 210 is provided in the second housing component 200. There are numerous further examples conceivable and within the disclosure of the present application, wherein the insert is provided on the second housing component and wherein the correspondingly-shaped receptacle is provided on the first housing component. Likewise, the specific implementation of radially protruding and radially recessed features, as described in connection with the mechanical coding, the mechanical counter coding or as described in connection with the protrusion and the groove or in connection with the fastening element and counter fastening element may be interchanged provided and implemented in an inverted way compared to the presently shown examples.

### REFERENCE NUMBERS

1 injection device
2 distal direction
3 proximal direction
4 dose incrementing direction
5 dose decrementing direction
6 cartridge
7 bung
8 drive mechanism
9 dose setting mechanism
10 housing
11 trigger
12 dose dial
13 dosage window
14 cartridge holder
15 injection needle
16 inner needle cap
17 outer needle cap
18 protective cap
20 piston rod
21 bearing
22 first thread
23 pressure foot
24 second thread
25 barrel
26 seal
28 threaded socket
30 drive sleeve
31 threaded section
32 flange
33 flange
35 last dose limiter
40 spring
60 clutch
62 insert piece
64 stem
80 number sleeve
81 groove
90 ratchet mechanism
91 ratchet feature
100 housing component
101 connecting end
102 sidewall
105 outside surface
108 indicator
110 insert
112 end face
114 abutment face
115 flange section
120 fastening element
121 snap element
122 recess
124 tongue portion
125 chamfer
126 slit
127 recess
130 groove
150 mechanical coding
151 coding feature
152 coding portion
200 housing component
201 connecting end
202 sidewall
203 inside surface
204 beveled surface section
205 outside surface
206 flat section
207 flat section
208 indicator
210 receptacle
211 insert opening
212 end face
214 end face
215 sidewall portion
218 indicator
220 counter fastening element
221 counter snap element
222 projection
225 recess
230 protrusion
231 chamfered section
250 mechanical counter coding
251 counter coding feature
252 counter coding portion
254 counter coding slot
350 mechanical coding
351 coding feature
352 coding recess
450 mechanical counter coding
451 counter coding feature
452 counter coding protrusion
550 mechanical coding
551 coding feature
650 mechanical counter coding
651 counter coding feature
750 mechanical coding
751 coding feature

1.-15. (canceled)

16. A housing of a drug delivery device, the housing comprising:
a first housing component configured to accommodate a cartridge filled with a medicament, the first housing component comprising a first connecting end,
a second housing component configured to accommodate a drive mechanism of the drug delivery device, the second housing component comprising a second connecting end,
an insert provided on one of the first connecting end and the second connecting end,
a receptacle provided on the other one of the first connecting end and the second connecting end, wherein the insert is insertable into the receptacle along a longitudinal direction for mutually fastening the first housing component and the second housing component,
a fastening element provided on the insert,
a counter fastening element complementary shaped to the fastening element and provided in the receptacle,
a groove provided on one of the insert and the receptacle and extending along the longitudinal direction,
a protrusion provided on the other one of the insert and the receptacle and configured to slide along the groove upon insertion of the insert into the receptacle thereby rotationally locking the first housing component relative to the second housing component,
a mechanical coding provided on the insert and comprising a coding feature,
a mechanical counter coding provided in the receptacle and comprising a counter coding feature,
wherein the mechanical coding and the mechanical counter coding are operable to prevent an engagement of the fastening element with the counter fastening element when the mechanical coding does not match the mechanical counter coding.

17. The housing according to claim 16, wherein the mechanical coding is defined by a cross-sectional geometry of a coding portion of a sidewall of the insert.

18. The housing according to claim 16, wherein the mechanical coding is defined by a cross-sectional geometry of a longitudinal end face of the insert.

19. The housing according to claim 17, wherein the cross-sectional geometry of the coding portion comprises one of a circular shape, an oval-shape, a triangular shape, a rectangular shape and a polygonal shape.

20. The housing according to claim 16, wherein the mechanical counter coding is defined by a cross-sectional geometry of a counter coding portion of a sidewall of the receptacle.

21. The housing according to claim 20, wherein the cross-sectional geometry of the counter coding portion comprises one of a circular shape, an oval shape, a triangular shape, a rectangular shape and a polygonal shape.

22. The housing according to claim 20, wherein the receptacle comprises an inner sidewall portion co-axial with the counter coding portion and forming a circumferential, longitudinally extending counter coding slot with the counter coding portion.

23. The housing according to claim 20, wherein the receptacle comprises an insert opening with a first cross section, wherein the counter coding portion is located longitudinally offset from the insert opening and comprises a second cross section,
wherein the second cross section is smaller than the first cross section, and
wherein an inside surface of the sidewall extending longitudinally from the insert opening towards the counter coding portion comprises a beveled surface section.

24. The housing according to claim 16, wherein the mechanical coding comprises at least one of a coding recess and a coding protrusion extending in the longitudinal direction, and wherein the mechanical counter coding comprises at least one of a counter coding recess matching with the coding protrusion and a counter coding protrusion matching with the coding recess.

25. The housing according to claim 16, wherein the fastening element comprises a snap element, and wherein the mechanical coding is defined by at least one of a longitudinal position and a longitudinal extent of the snap element on the insert.

26. The housing according to claim 16, wherein the counter fastening element comprises a counter snap element, and wherein the mechanical counter coding is defined by at least one of a longitudinal position and a longitudinal extent of the counter snap element in the receptacle.

27. The housing according to claim 16, wherein the receptacle comprises a second counter fastening element located diametrically opposite to the counter fastening element, and wherein a sidewall of the receptacle is elastically deformable to increase a radial distance between the counter fastening element and the second counter fastening element, wherein the radial distance is larger than or equal a radial distance between the fastening element and a second fastening element located diametrically opposite on the insert.

28. The housing according to claim 27, wherein the sidewall of the receptacle comprises an outside surface, the outside surface comprising a first flat section and a second flat section radially opposite the first flat section, wherein a first imaginary straight line intersecting the first flat section and the second flat section extends substantially perpendicular to a second imaginary straight line intersecting the counter fastening element and the second counter fastening element.

29. An injection device for injecting a dose of a medicament, the injection device comprising:
a housing comprising:
a receptacle provided on an end of the housing and including a counter fastening element and a mechanical counter coding with a counter coding feature;
an insert provided on a different end of the housing and being insertable into the receptacle along a longitudinal direction, the insert including a fastening element and a mechanical coding with a coding feature;
wherein the counter fastening element is complementary shaped to the fastening element; and
wherein the mechanical coding and the mechanical counter coding are operable to prevent an engagement of the fastening element with the counter fastening element when the mechanical coding does not match the mechanical counter coding;
a cartridge arranged inside the housing, the cartridge comprising a barrel filled with a medicament and sealed in a proximal longitudinal direction by a movable bung; and
a drive mechanism arranged inside the housing, the drive mechanism comprising a piston rod operable to exert a distally directed dispensing force onto the bung of the cartridge.

30. The injection device of claim 29, comprising a groove provided on one of the insert and the receptacle and extending along the longitudinal direction.

31. The injection device of claim 30, comprising a protrusion provided on the other one of the insert and the receptacle and configured to slide along the groove upon insertion of the insert into the receptacle.

32. The injection device of claim 31, wherein the housing comprises:
a first housing component configured to accommodate the cartridge filled with a medicament, the first housing component comprising the end of the housing,
a second housing component configured to accommodate the drive mechanism, the second housing component comprising the different end of the housing;
wherein the first housing component is rotationally locked relative to the second housing component when the insert is inserted into the receptacle.

33. An injection device kit comprising:
a first housing and a second housing, wherein each of the first and second housing comprises:
a first connecting end;
a second connecting end;
a receptacle provided on the first or second connecting end, the receptacle including a counter fastening element and a mechanical counter coding with a counter coding feature;
an insert provided on the other of the first or second connecting end, the insert being insertable into the receptacle, and including a fastening element and a mechanical coding with a coding feature;
wherein the counter fastening element is complementary shaped to the fastening element; and
wherein the mechanical coding and the mechanical counter coding are operable to prevent an engagement of the fastening element with the counter fastening element when the mechanical coding does not match the mechanical counter coding; and
wherein the coding feature of the first housing distinguishes from the coding feature of the second housing with regard to at least one of:
a number of coding features,
a longitudinal position,
a longitudinal extent,
a circumferential position,
a circumferential extent,
a cross-sectional geometry or shape in a plane transverse to a longitudinal direction.

34. The injection device kit of claim 33, wherein each of the first and second housing comprises a groove provided on one of the insert and the receptacle and extending along a longitudinal direction.

35. The injection device kit of claim 34, wherein each of the first and second housing comprises a protrusion provided on the other one of the insert and the receptacle and configured to slide along the groove upon insertion of the insert into the receptacle.
